(19) United States
(12) Patent Application Publication
(10) Pub. No.: US 2004/0148024 A1
(43) Pub. Date: Jul. 29, 2004

(54) Soft tissue implant

(76) Inventor: David F. Williams, Kingsley (GB)

Correspondence Address: Drinker Biddle & Reath, One Logan Square, 18th & Cherry Streets, Philadelphia, PA, 19103-6996, US

(21) Appl. No.: 10/450,434

(22) PCT Filed: Dec. 17, 2001
(86) PCT No.: PCT/GB01/05575

(30) Foreign Application Priority Data
Dec. 15, 2000 (GB) 0030635.7

(51) Int. Cl. A61F 2/12

(52) U.S. Cl. 623/8; 623/11.11; 623/23.72

(57) ABSTRACT

The present invention provides a soft tissue implant comprising an outer shell including at least one layer of biostable impervious polyurethane, wherein the outer surface of the outer shell comprises undulations to encourage tissue adaptation to the implant whilst preventing tissue ingrowth and wherein the implant is gel free. The outer shell may comprise multiple concentric layers and a core of polymer fabricated as a closed cell foam.

The present invention relates to soft tissue implants. In particular, the invention relates to mammary implants and to the use of particular materials for soft tissue implants.

Breast implants, sometimes known as mammary implants, have been used for the reconstruction or augmentation of breasts for over 50 years. Early attempts to augment the female breast can be dated to the 1940's and 50's, when either injection of fluids such as silicone or paraffin oils, or various sponges such as polyvinyl alcohol, were used. Neither of these approaches were successful. The discrete injection of fluids, sometimes of an undefined chemical nature, elicited unacceptable tissue reactions, whilst the use of completely porous structures resulted in extensive tissue ingrowth that caused calcification and unacceptable hardness and disfigurement. A breast implant ideally should replicate the consistency, resilience and elasticity of natural breast tissue, whilst recognising that these characteristics do vary from individual to individual. There is no homogenous synthetic material that is able to replicate these characteristics and which is also capable of fabrication into a defined shape. The first breast implant of clinical and commercial value was introduced in 1962 and overcame this difficulty by taking a viscous gel that had many of these desirable characteristics, in this case a silicone gel, and encapsulating this gel in a silicone elastomer envelope or shell. Such implants, the so-called silicone gel-filled silicone breast implants have been used in many patients over the last forty years. A number of problems occur with these implants however such that currently there is considerable regulatory and clinical reluctance to use them. The first significant problem to be recognised was that of constrictive fibrous, wherein the fibrous capsule that normally forms around an implant became significantly thicker, causing contracture with resulting pain and disfigurement. This problem was largely resolved clinically by rendering the surface of the implant with a texture or roughness that allowed a certain degree of tissue ingrowth. This stabilised the implant and minimised the tendency of fibroblasts to produce excessive collagen, creating the thicker capsule. The preferred embodiment of this concept, as described in the patents of Pangman was a polyurethane foam, specifically a polyester urethane foam. However, this foam was eventually found to degrade and since one of the degradation products was a suspect carcinogen, this use was discontinued. Other implants have attempted to utilise a textured silicone structure on the outer surface of the silicone elastomer.

Two further problems have occurred with the silicone gel-filled breast implants. Any elastomer, which by definition is flexible, may be permeable to certain molecules. The silicone elastomer is weakly permeable to the silicone gel that it contains, especially with respect to the lower molecular weight components of the gel, such that a small amount, typically less than 1% of the gel diffuse out through the envelope. The fate of these diffusible gel components has been controversial, but has been instrumental in causing adverse clinical and patient opinion of these implants.

The second problem has been that the elastomer shell has a tendency to rupture. This can occur under several conditions, principally involving trauma to the chest. The consequences of this rupture are also controversial but a rupture can lead to loss of shape of the implant and the release of significant volumes of silicone gel into the tissue.

One attempt to resolve these problems with the silicone gel has been to employ a saline solution as the filler of the implant. This has been moderately successful but the saline, because of its fluidity, cannot provide the cohesiveness required to give the implants reasonable shape. An alternative solution has been to provide a triglyceride oil filler, specifically soya bean oil with a declared advantage of a natural substance, the leakage of which should not have been problematic biologically. However, the oxidative ageing of this oil, potentially leading to adverse tissue effects, has negated this advantage and these implants are no longer available.

The result of this sequence of events has been that, in spite of a significant demand for augmentation and reconstruction prostheses, no acceptable form is currently available. The demand can be reliably measured as hundreds of thousands of patients per year, meeting physical and psychological needs.

It is an aim of the present invention to provide a breast implant that does not involve a gel filler. All of the problems with previous breast implants over the last 50 years have been related to the presence of gels, and the potential release of these gels through bleed or rupture, and the adverse biological consequences of such release.

The problems mentioned herein in relation to breast implants also have relevance in relation to other soft tissue implants designed to replace and augment tissues chosen from but not limited to testicular tissue, cartilage, muscle and any connective tissue apart from teeth and bones.

Accordingly, the present invention provides a soft tissue implant, the implant comprising an outer shell including at least one impervious layer of a biostable polyurethene and not including gel.

Preferably the shell comprises more than one impervious layer wherein at least the outer layer of the shell is of a biostable polyurethane. The layers may be the same or different and preferably concentric. Additional layers may be silicon or teflon or combination thereof.

The implant may include a filler, the filler comprising any solid polymer fabricated as a closed cell foam. This enables the compliance and elasticity to be achievable by using an elastomeric microporous structure that does not require a gel filling.

Preferably the material of this microporous structure is derived from an elastomer that has been designed to possess a unique combination of biostability and compliance that will allow for the replication of soft tissue texture and resilience.

Preferably the implant will have an external surface with a topography that will facilitate tissue adaptation, thus minimising capsular contracture but not encouraging tissue ingrowth, thus preventing any inflammatory response that would aggravate the aggressiveness of the tissue environment.

Although one embodiment of the invention relates to a breast implant it is also intended that the invention should be amenable to any soft tissue augmentation or reconstruction device including, but not limited to, all forms of soft tissue maxillofacial devices used for example in the reconstruction of the nose, chin and zygoma, sphincter augmentation such as in the urinary and gastrointestinal systems, penile implants and testicular replacements and cosmetic muscular enlargement.

The term soft tissue includes all connective tissue apart from bones and teeth.

A breast implant according to one aspect of the invention may be of any size and shape to suit the requirements of any individual patient but will typically have a circular base with a shallow bowl-shaped body and a volume of between 50 and 750 cubic centimetres. The interior of the body of the implant will consist of a microporous structure, the volume fraction, orientation and size of the pores varying according to the compliance and resilience required of the particular device. Preferably the microporous structure is based on polyurethane or silicon or a mixture thereof. The microporous structure may be a foam. In order to achieve differential resistance to deformation in different directions the microporous structure may be arranged with a series of directional supports or septae comprising an appropriate elastomeric material.

The outer shell of the breast implant may comprise a plurality of layers. Preferably the shell comprises two non-porous, possibly interconnected layers, the interconnection being of a widely spaced honeycomb structure giving maximal resistance to compression of this outer shell, the double layer of impermeable elastomer maximising resistance to inward diffusion of body fluids.

The outer of these two layers in the shell shall preferably have a smooth inner surface and an undulating outer surface. The minimum thickness of this layer shall be at least 50 μm, preferably at least 100 μm with undulations of amplitude in the region of 50 to 500 microns, and distance between peaks in the region of 500 microns to 5 mm. The thickness of the gap between the two outer layers shall be between 50 and 500 microns and the thickness of the inner layer shall be up to 2 mm.

The undulations assist tissue adaptation and holding of the implant without tissue intrusion and thickening. Use of the biostable polyurethane together with undulating surface topography maximises tissue adaptation to the implant.

The core structure of the implant shall be made of a suitable elastomeric material, preferably but not limited to a biostable polyurethane, alternatives including silicone elastomers. Whereas, the preferred core material may be an ElastEon polyurethane, the core may consist of any solid polymer which can be fabricated as a closed cell foam. Suitable polymers include polyethylene, polypropylene or any other polyurethane.

Whereas polyurethanes are susceptible to degradation the ELASTEON™ polymers have been designed to minimise degradation The layers of the outer shell, including, for example, a honeycomb spacer, shall be made of a biostable polyurethane, preferably this material shall be one of the Elast-Eon family of polyurethanes.

In certain implants it may be desirable to have structural elements. Preferably the structural elements are manufactured from the same material as the outer shell of an implant. They may be made of other materials. Structure and position of structural elements depends on size and type of implant.

The porosity of the core material will be dependent on the use of the soft tissue implant, for example cartilage would be low porosity whereas breast tissue would be higher porosity. Pores could range from 10 μm to 5 mm diameter.

Most preferably the exterior surface of an implant according to the present invention is a member of the family of polyhexamethylene oxide based aromatic polyurethanes of hardness ranging from 80A-75D (shore), for example, ELASTEON™ 1, or one of the family of siloxane based macrodial aromatic polyurethanes of hardness from 80A to 55D, for example ELASTEON™ 2, or any siloxane based macrodial, modified hard segment aromatic polyurethane of hardness 65A to 80A, for example ELASTEON™ 3.

This concept is not limited to breast implants and may be adapted to other forms of implantable device used for the augmentation of soft tissues. In each case the architecture and anisotropy of the core of the implant will be varied in order to match the characteristics of the tissue that is subject to augmentation. Examples include reconstruction of the outer ear, alteration to the shape of the nose, modification to the soft tissue coverage of the mandible including the cheek and the chin, correction of deformities or trauma in the orbit of the eye, any cosmetic intramuscular device, devices to alter the shape of the larynx to treat vocal chord injury, treatment of lax sphincter muscles at the base of the bladder and in the rectum, penile implants, testicular replacement and implants in the inter-vertebral disc space.

The invention is exemplified with reference to the accompanying figures, wherein:

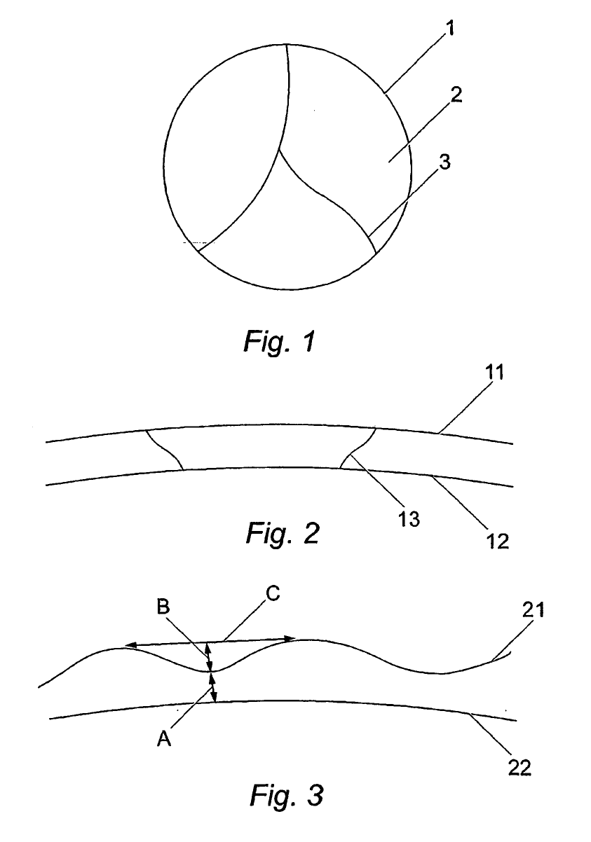

FIG. 1 illustrates the structure of a breast implant.

FIG. 2 illustrates the outer shell of the implant.

FIG. 3 illustrates the surface topography of an implant according to the invention.

The invention uses an impervious outer layer of a biostable polyurethane to prevent ingrowth from tissue when implanted. Also, the surface morphology of the outer layer is designed to minimise capsule formation by being macroscopically undulating and microscopically smooth.

A breast implant according to one aspect of the invention as shown diagramatically in FIG. 1, may be of any size and shape to suit the requirements of any individual patient but will typically have a circular base with a shallow bowl-shaped body and a volume of between 50 and 750 cubic centimetres. The outer shell (1) will comprise at least one layer of impervious polyurethane. The interior of the body of the implant will consist of a microporous structure (2), the volume fraction, orientation and size of the pores varying according to the compliance and resilience required of the particular device. Preferably the microporous structure is based on polyurethane or silicon or a mixture thereof. The microporous structure may be a foam. In order to achieve differential resistance to deformation in different directions the microporous structure may be arranged with a series of directional supports or septae (3) comprising an appropriate elastomeric material.

As shown in FIG. 2, the outer shell of the breast implant may comprise a plurality of layers. Preferably the shell comprises two non-porous, possibly interconnected layers (11,12), the interconnection (13) being of a widely spaced honeycomb structure giving maximal resistance to compression of the outer shell.

As shown in FIG. 3, the outer of the layers of the shell has a smooth inner surface (22) and an undulating outer surface (21). The minimum thickness (A) is at least 50 μm with undulations in omplitude (B) of between 50 to 50 μm.

The distance between peaks (C) is in the region of 500 μm to 5 mm. Preferably the implant uses ElastEon polymers as available from AorTech Biomaterials.

Accordingly the invention comprises the use of biostable polyurethanes in the production of soft tissue implants.

1. A soft tissue implant comprising an outer shell of a biostable impervious polyurethane.

2. A soft tissue implant as claimed in claim 1 wherein the outer shell comprises at least two concentric layers wherein at least one layer consists of a biostable impervious polyurethane.

3. A soft tissue implant as claimed in claim 1 or claim 2 wherein at least one layer of the outer shell has a smooth inner surface and an undulating outer surface, wherein the undulating outer surface is microscopically smooth and macroscopically undulating.

4. A soft tissue implant as claimed in any of the preceding claims wherein the implant comprises a core of polymer fabricated as a closed cell foam.

5. A soft tissue implant as claimed in any of the preceding claims wherein the implant includes internal structural elements.

6. A soft tissue implant as claimed in any of the preceding claims which is a breast implant.

7. Use of biostable impervious polyurethanes in a soft tissue implant as claimed in any of the preceding claims.

8. Use of a member of the family of polyhexamethylene oxide based aromatic polyurethanes of hardness ranging from 80A-75D (shore), one of the family of siloxane based macrodial aromatic polyurethanes of hardness from 80A to 55D, or any siloxane macrodial, modified hard segment aromatic polyurethane of hardness 65A to 80A in a soft tissue implant as claimed in any of the preceding claims.

9. A soft tissue implant comprising an outer shell including at least one layer of biostable impervious polyurethane, wherein the outer surface of the outer shell comprises undulations to encourage tissue adaptation to the implant whilst preventing tissue ingrowth and wherein the implant is gel free.
